(19) United States
(12) Patent Application Publication
(10) Pub. No.: US 2005/0155815 A1
(43) Pub. Date: Jul. 21, 2005

(54) Standing wave absorbing device for vehicle

(75) Inventors: Katsuhiro Sasaki, Yamagata (JP); Toshihiro Hikichi, Yamagata (JP); Takayuki Negishi, Yamagata (JP); Yasuhisa Abe, Yamagata (JP); Takeshi Ito, Yamagata (JP); Hiroyuki Hamada, Saitama (JP); Shintaro Hosoi, Saitama (JP)

Correspondence Address: SUGHRUE MION, PLLC, 2100 PENNSYLVANIA AVENUE, N.W., SUITE 800, WASHINGTON, DC, 20037, US

(73) Assignees: PIONEER CORPORATION; TOHOKU PIONEER CORPORATION

(21) Appl. No.: 10/989,603

(22) Filed: Nov. 17, 2004

(30) Foreign Application Priority Data
Nov. 17, 2003 (JP) P2003-286148

(51) Int. Cl. H05K 5/00; A61F 11/06; G10K 11/16; H03B 29/00

(52) U.S. Cl. 181/151; 381/71.5

(57) ABSTRACT

A standing wave absorbing device for a vehicle, includes a sound tube provided at a predetermined position within a compartment or inside a door. The sound tube absorbs unwanted standing wave occurring within the compartment or inside the door.

### BACKGROUND OF THE INVENTION

1. Field of the Invention

The present invention relates to a standing wave absorbing device for absorbing unwanted standing wave occurring within a compartment or inside a door for a vehicle.

2. Description of the Related Art

The compartment of a vehicle is a closed narrow space. If entering the compartment, there may be a unique feeling or a sense of oppression. This feeling or sense is involved in the standing wave caused by the vehicle interior structure or compartment structure, and is relieved only by opening a window glass to change the atmosphere. That is, the compartment is a unique closed narrow space, unlike the general room.

This standing wave will be detailed below. When the vehicle is running, the running noise such as road noise or engine sound occurs. Within the vehicle, this running noise is augmented by the standing wave caused by the vehicle interior structure or compartment structure. This standing wave has a frequency of about 100 Hz mainly caused by the vehicle width, or a frequency of about 150 Hz caused by the vehicle height, and has a property of lasting for a long time as the reverberation. Therefore, if the driver or fellow passenger is stimulated by the standing wave persistently, the driver has a driving fatigue, or the fellow passenger induces a car sickness due to depression of a semicircular canal caused by the fatigue.

Conventionally, to remove or decrease the standing wave within the vehicle, an electric processing was conducted (for example, JP-A-2000-261879).

JP-A-2000-261879 discloses that the acoustic characteristics are measured by a microphone placed at any position in the compartment, the measured values are analyzed, and the standing wave is controlled by changing the position or direction of the speaker, based on the analyzed values.

However, the conventional technique involves measuring the acoustic characteristics in the compartment containing the standing wave, employing the microphone, and making the electric processing and the analysis to reduce the standing wave, and is complex and expensive in its constitution.

### SUMMARY OF THE INVENTION

It is an object of the invention to provide a standing wave absorbing device for decreasing unwanted standing wave occurring in a space within the compartment or inside the door for the vehicle with a simple constitution, and reducing or resolving an adverse physiological influence such as fatigue or car sickness caused by this standing wave.

It is another object of the invention to provide a standing wave absorbing device in which the acoustic characteristics of the compartment is improved by decreasing the standing wave occurring within the compartment or inside the door.

According to a first aspect of the invention, a standing wave absorbing device for a vehicle, includes a sound tube provided at a predetermined position within a compartment or a door, the sound tube absorbing unwanted standing wave occurring within the compartment or the door.

### BRIEF DESCRIPTION OF THE DRAWINGS

FIG. 1 is a view showing the relationship between the selected absorption frequency of a sound tube and the resonance tube length (sound tube length) according to an embodiment of the present invention.

FIG. 2 is a view showing the constitution of the sound tube according to an embodiment of the invention.

FIG. 3 is a view showing a standing wave absorbing device according to an embodiment of the invention.

FIG. 4 is a chart showing the frequency characteristic of the standing wave absorbing device according to an embodiment of the invention.

FIG. 5 is a chart showing the reverberation characteristic of the standing wave absorbing device according to an embodiment of the invention.

FIGS. 6A and 6B are views showing the standing wave absorbing device according to an embodiment of the invention.

FIG. 7 is a chart showing the frequency characteristic of the standing wave absorbing device according to an embodiment of the invention.

FIG. 8 is a chart showing the reverberation characteristic of the standing wave absorbing device according to an embodiment of the invention.

FIG. 9 is a view showing the standing wave absorbing device according to an embodiment of the invention.

FIG. 10 is a view showing the standing wave absorbing device according to an embodiment of the invention.

FIG. 11 is a view showing the standing wave absorbing device according to an embodiment of the invention.

FIGS. 12A and 12B are views showing the standing wave absorbing device according to an embodiment of the invention.

FIGS. 13A and 13B are views showing the standing wave absorbing device according to an embodiment of the invention.

FIG. 14 is a view showing the sound tube according to another embodiment of the invention.

FIGS. 15A and 15B are views showing the sound tube according to another embodiment of the invention.

### DETAILED DESCRIPTION OF THE PREFERRED EMBODIMENTS

The present invention will be described below. To easily understand the invention, reference numerals are attached in the accompanying drawings. However, the invention is not limited to the following embodiments.

In order to achieve the above objects, according to claim 1 of the invention, there is provided a standing wave absorbing device for a vehicle, characterized in that a sound tube 1 for absorbing unwanted standing wave occurring within a compartment or inside a door for the vehicle is provided at a predetermined position within said compartment or inside said door.

Before explaining the specific embodiments, first of all, a sound tube according to the present invention will be described below.

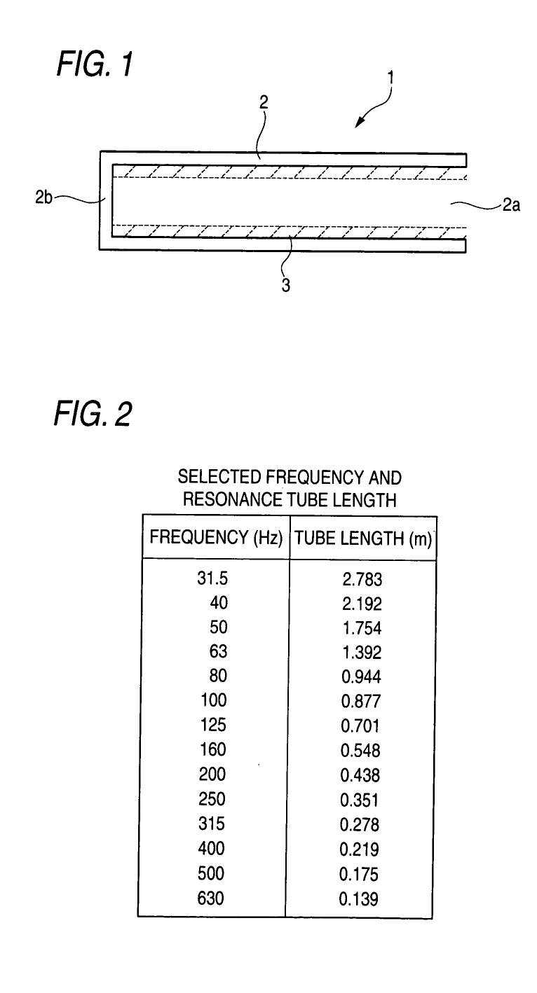

FIG. 1 is a cross-sectional view showing a structure of the sound tube 1. The sound tube 1 comprises a sound tube portion 2, and a sound absorbing material 3 (acoustic resistor) formed by flocking on an inner wall portion of the sound tube portion 2. The sound tube portion 2 has an opening portion 2a opened at one end, and a closed end portion 2b closed at the other end. Employing the resonance of a quarter wavelength tube of the sound tube portion 2, an inside of the sound tube is damped by the sound absorbing material 3 to constitute a closed tube of quarter wavelength to selectively absorb the sound at a specific frequency. The length of the sound tube portion 2 (resonance tube length) for selectively absorbing the sound at the specific frequency, employing the resonance of the quarter wavelength tube, is given by the following formula.
Resonance tube length L(m)=sound speed (334 m)÷selective sound absorbing frequency (Hz)÷4×1.05
Where the multiplier 1.05 is a correction factor for the inserted sound absorbing material 3.

FIG. 2 shows the relationship between the selected absorption frequency and the resonance tube length (length of the sound tube 1) as obtained by the above formula. For example, the tube length is 0.877 m to absorb a standing wave of about 100 Hz produced by the vehicle width, and the tube length is 0.548 m to absorb a standing wave of about 150 Hz (standing wave of 160 Hz) produced by the vehicle height.

The sound absorbing material 3 may use glass wool, natural wool, man-made wool, or man-made and natural wool, for example.

An embodiment of a sound absorbing device for unwanted standing wave according to the invention will be described below with reference to the drawings.

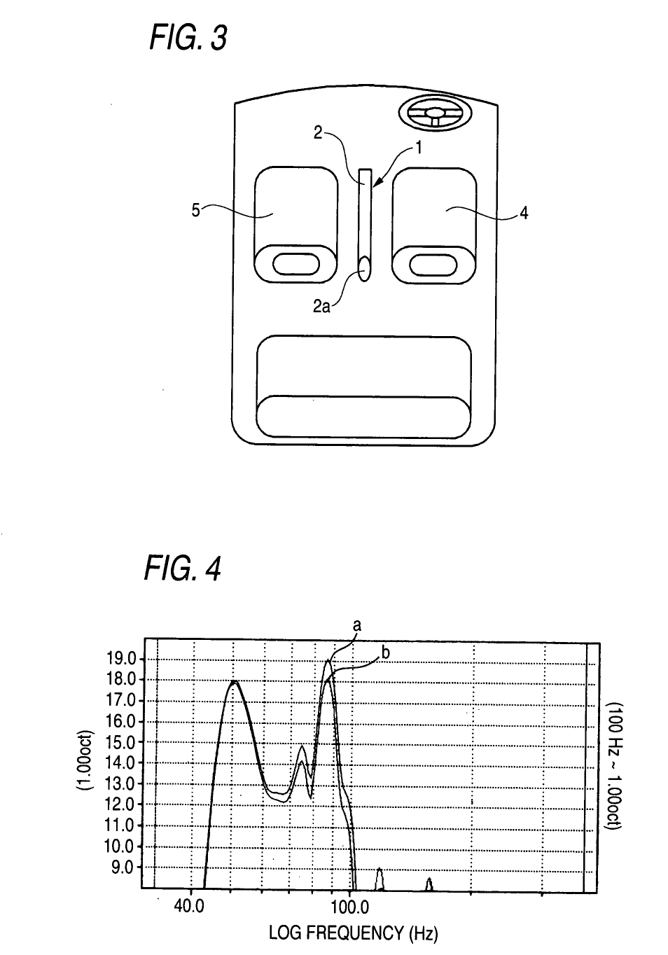

FIG. 3 is a view showing the standing wave absorbing device according to the embodiment of the invention, as the vehicle interior is seen from above.

The sound tube 1 is installed between a driver's seat 4 and an assistant driver's seat 5 to decrease the standing wave from 80 Hz to 100 Hz involving the vehicle width. At this time, the sound tube 1 is disposed so that the opening portion 2a of the sound tube portion 2 may be on the rear side.

FIG. 4 shows the sound damping characteristic for the frequency according to the embodiment as shown in FIG. 3. In FIG. 4, when the sound tube 1 is not installed, the characteristic a is indicated, while when the sound tube 1 is installed, the characteristic b is indicated. It will be found that the sound pressure is damped at about 80 Hz to 100 Hz.

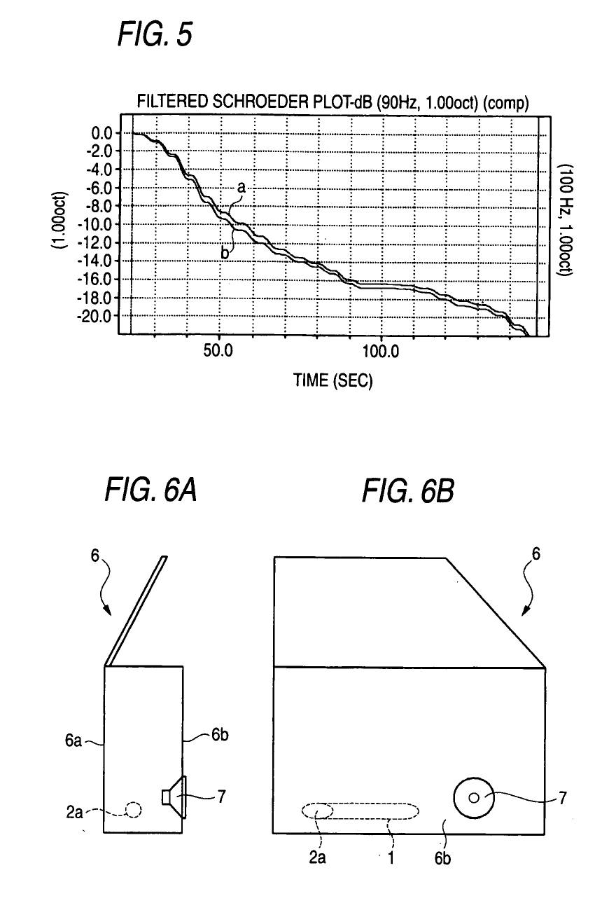

FIG. 5 shows the reverberation characteristic according to the embodiment as shown in FIG. 3. In FIG. 5, the characteristic a is indicated when the sound tube 1 is not installed. When the sound tube 1 is installed, the characteristic b is indicated and the reverberation characteristic is improved. The opening portion 2a of the sound tube portion 2 may be placed at another location, such as on the front side, driver's seat or assistant driver's seat. In essence, it is required that the opening portion 2a is placed so that the standing wave may be damped between the driver's seat 4 and the assistant driver's seat 5 most effectively. According to this embodiment, it is possible to reduce or resolve the physiological phenomenon such as driving fatigue of the driver or car sickness of a fellow passenger due to unwanted standing wave.

FIGS. 6A and 6B are views showing the standing wave absorbing device according to another embodiment of the invention, in which FIG. 6A is a side view of a door portion and FIG. 6B is a front view thereof.

The door 6 of the vehicle comprises an outer panel 6a and an inner trim 6b, a speaker 7 being mounted on the inner trim 6b. The inner trim 6b is attached between the outer panel 6a and an inner panel which is not shown in FIGS. 6A and 6B. A space formed by the outer panel 6a and the inner trim 6b is aback cavity of the speaker 7. Also, the sound tube 1 including the opening portion 2a of the sound tube portion 2 is installed at arbitrary position in this space. When the speaker 7 is driven, a standing wave of e.g., about 80 Hz accordingly occurs in a space inside the door formed by the outer panel 6a and the inner trim 6b. The sound tube 1 absorbs the sound of this standing wave.

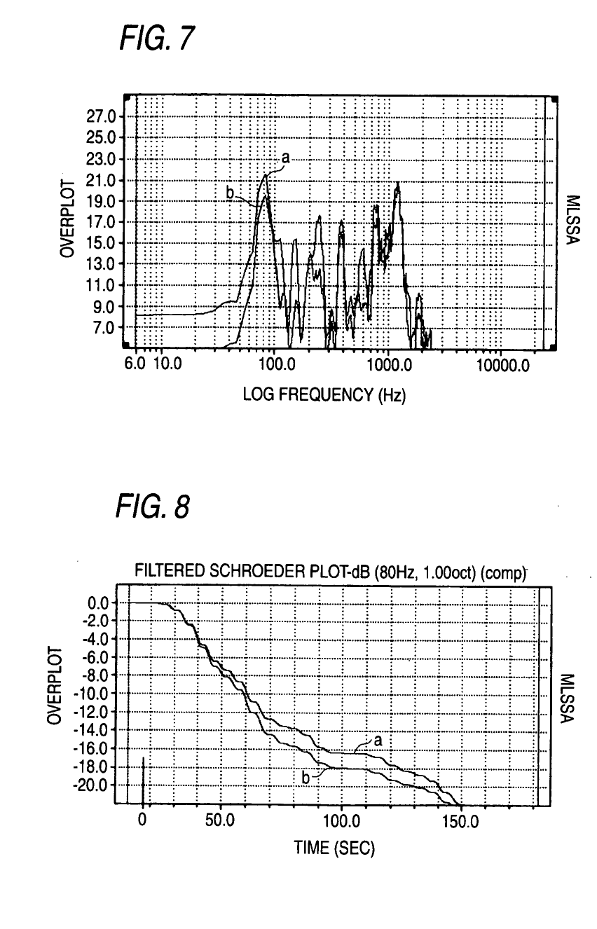

FIG. 7 shows the sound damping characteristic for the frequency according to the embodiment of FIG. 6. In FIG. 7, when the sound tube 1 is not installed, the characteristic a is indicated, while when the sound tube 1 is installed, the characteristic b is indicated. It will be found that the sound pressure is damped at about 80 Hz. FIG. 8 shows the reverberation characteristic according to the embodiment of FIG. 6. In FIG. 8, the characteristic a is indicated when the sound tube 1 is not installed, while the characteristic b is indicated when the sound tube 1 is installed. It will be found that the reverberation characteristic is improved. Since the vibration of the inner trim 6b or the inner panel, not shown, making up the door 6 is suppressed, unwanted radiation sound from the door 6 is decreased. Thereby, the sound quality of reproduced sound reproduced from the speaker 7 mounted on the door 6 is improved. The sound tube 1 may be molded integrally with a structure body making up the door 6.

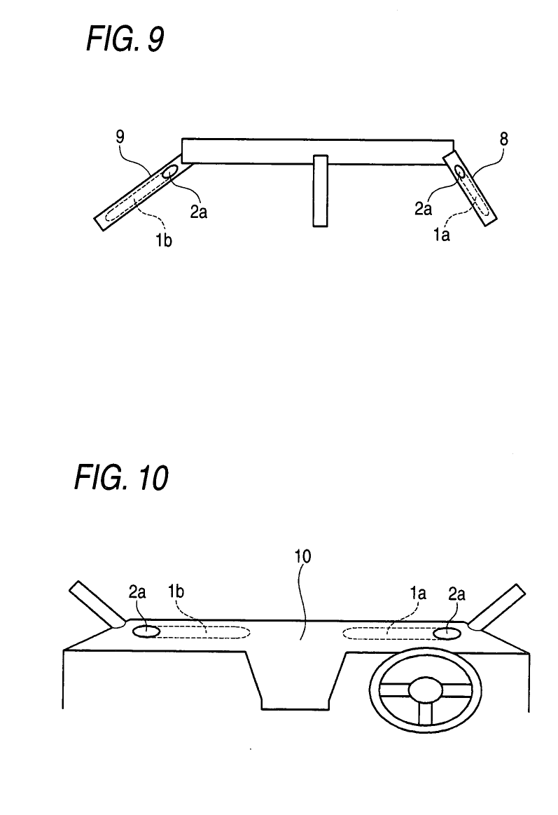

FIG. 9 shows the standing wave absorbing device according to another embodiment of the invention, and is a side view of the pillar portion comprising the front pillar and the rear pillar.

The sound tube 1a is contained in the front pillar 8 for the vehicle, and the sound tube 1b is contained in the rear pillar 9. The opening portions 2a and 2b of the sound tubes 1a and 1b are exposed to the interior of the compartment and absorb the standing wave of the compartment. The sound tubes 1a and 1b may be contained on both sides of the front pillar and the rear pillar to damp the standing wave more effectively.

FIG. 10 is a view showing the standing wave absorbing device according to another embodiment of the invention, and is a front view near the front panel.

One pair of left and right sound tubes 1a and 1b are attached inside the front panel 10 in the compartment. The opening portions 2a and 2b of the sound tubes 1a and 1b are located in the direction separating apart from each other, and exposed to the compartment to be suitable for absorbing the standing wave. The sound tubes may be contained on a rear board, not shown, in the compartment to expose the opening portions to the compartment.

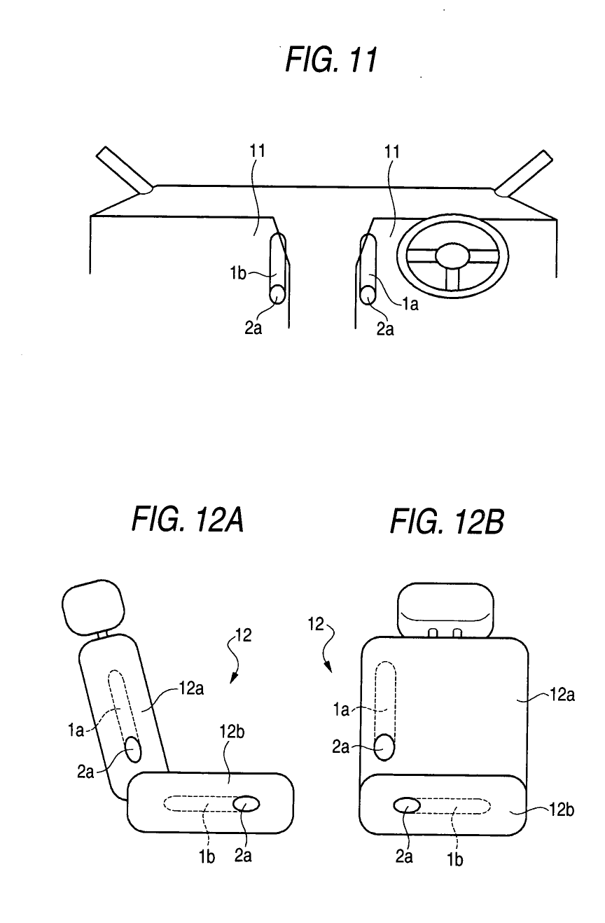

FIG. 11 is a view showing the standing wave absorbing device according to another embodiment of the invention, and showing a front view near a kick panel.

The sound tubes 1a and 1b are disposed on the kick panels 11 for the driver's seat and the assistant driver's seat in the compartment to absorb the standing wave near 300 Hz occurring on the kick panels 11.

FIGS. 12A and 12B are views showing the standing wave absorbing device according to another embodiment of the invention, in which FIG. 12A is a side view of the seat and FIG. 12B is a front view thereof.

The sound tubes 1a and 1b are contained on a seat back portion 12a and a seat portion 12b for the seat 12 of the vehicle, with the opening portions 2a of both the sound tubes being exposed to the interior of the vehicle. The sound tube 1a absorbs the standing wave caused by the vehicle height, and the sound tube 1b absorbs the standing wave caused by the vehicle width.

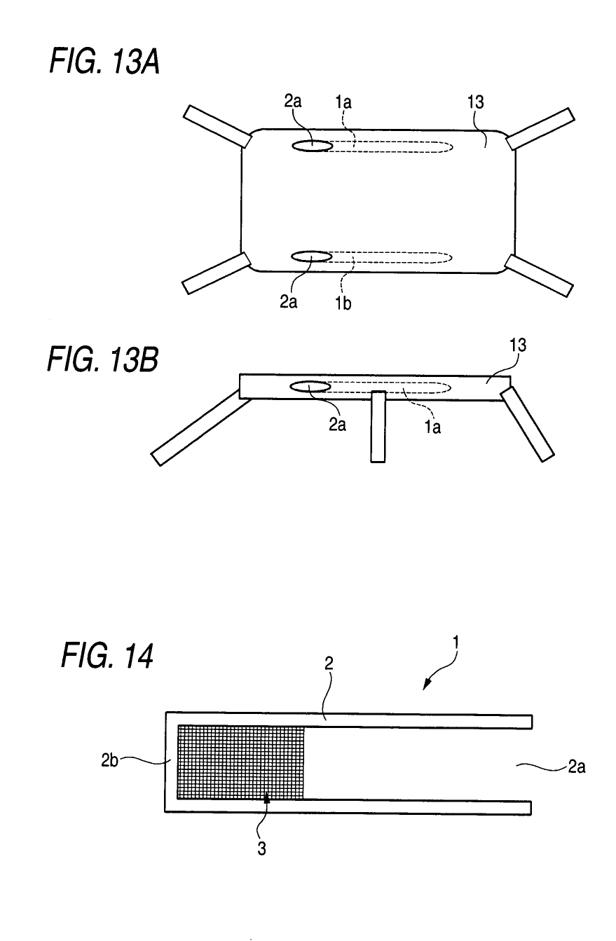

FIGS. 13A and 13B are views showing the standing wave absorbing device according to another embodiment of the invention, in which FIG. 13A is a plan view of the roof portion as seen from the inside of the vehicle, and FIG. 13B is a side view thereof.

One pair of sound tubes 1a and 1b that are bilateral in the running direction of the vehicle are contained in the roof portion 13 of the compartment, in which the opening portions 2a of both the sound tubes are exposed from the roof portion 13 to the interior of the vehicle. Thereby, the standing wave caused by the vehicle height is absorbed.

FIG. 14 is a cross-sectional view showing the sound tube according to another embodiment of the invention.

The sound absorbing material 3 is filled inside the closed portion 2b of the sound tube portion 2 to damp the standing wave entering through the opening portion 2a.

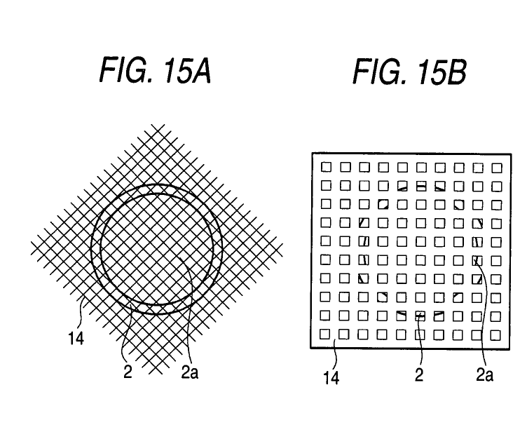

FIGS. 15A and 15B are views showing the opening portion of the sound tube according to another embodiment of the invention. To prevent foreign matter such as dirt or dust from entering through the opening portion 2a of the sound tube portion 2 into the inside, a foreign matter admission preventing member 14 is attached around the opening portion 2a. FIG. 15A shows an example in which a mesh or net member is employed as the foreign matter admission preventing member 14, and FIG. 15B shows an example in which a lattice member is employed.

The invention is not limited to the above embodiments, but may be modified in the following way. The sound tube may be attached within a trunk room to absorb the standing wave occurring within the trunk room.

Though a straight tube is employed as the sound tube 1 in the above embodiments, a curved tube or a tube having any other shape may be employed.

Though the sound tube 1 and the structural member of the vehicle are separately provided in the above embodiment, but maybe integrally formed, or the structural member of the vehicle itself may be employed as the sound tube.

Though the arrangement of sound tubes is described in terms of the representative locations in the above embodiments, the sound tubes may be arranged at other locations, such as floor of the vehicle, rear seat, and center pillar.

1. A standing wave absorbing device for a vehicle,
comprising:
a sound tube provided at a predetermined position within a compartment or a door, the sound tube absorbing an unwanted standing wave occurring within the compartment or and the door.

2. The standing wave absorbing device according to claim 1, wherein the sound tube comprises a sound tube portion and an acoustic resistor fitted into the sound tube portion, and the sound tube portion has an opening portion at one end of the sound tube portion and a closing portion at the other end.

3. The standing wave absorbing device according to claim 1, wherein the unwanted standing wave is caused by a vehicle width of the vehicle, a vehicle height of the vehicle, a kick panel of a driver's seat or a kick panel of an assistant driver's seat.

4. The standing wave absorbing device according to claim 1, wherein the unwanted standing wave occurs within the door by driving a speaker which is mounted on the door.

5. The standing wave absorbing device according to claim 1, wherein the sound tube is provided separately from vehicle components making up the vehicle.

6. The standing wave absorbing device according to claim 1, wherein the sound tube is integrally formed with vehicle components making up the vehicle.

7. The standing wave absorbing device according to claim 2, wherein the sound tube is contained in at least one of a seat portion of a driver's seat, a seat back portion of the driver's seat, a seat portion of an assistant driver's seat and a seat back portion of the assistant driver's seat, and the opening portion is exposed.

8. The standing wave absorbing device according to claim 2, wherein the sound tube is contained in a roof portion and the opening portion is exposed.

9. The standing wave absorbing device according to claim 4, wherein the sound tube is contained inside the door.

10. The standing wave absorbing device for according to claim 2, wherein the sound tube is contained in a front pillar or a rear pillar, and the opening portion is exposed.

11. The standing wave absorbing device according to claim 2, wherein the sound tube is contained in a front panel, and the opening portion is exposed on an upper part of the front panel.

12. The standing wave absorbing device according to claim 2, wherein the sound tube is contained in a front panel, and the opening portion is located in a lower part of the front panel and directed toward at least one of a driver's seat and an assistant driver's seat.

13. The standing wave absorbing device according to claim 1, wherein the sound tube is between a driver's seat and an assistant driver's seat.

14. The standing wave absorbing device according to claim 2, wherein a foreign matter preventing member is provided at the opening portion.

15. The standing wave absorbing device according to claim 1, wherein a sound absorbing material is provided inside the sound tube.

16. The standing wave absorbing device according to claim 15, wherein a sound absorbing material is provided on an inner wall face of the sound tube.
